(19) United States
(12) Patent Application Publication
(10) Pub. No.: US 2024/0279247 A1
(43) Pub. Date: Aug. 22, 2024

(54) PRODRUGS OF BORON COMPOUNDS AND THEIR USE IN TREATING BACTERIAL INFECTIONS

(71) Applicant: SHANGHAI MICURX PHARMACEUTICALS CO., LTD., New District (CN)

(72) Inventors: Xinghai Wang, Shanghai (CN); Jinqian Liu, Fremont, CA (US); Mikhail Fedorovich Gordeev, Castro Valley, CA (US)

(21) Appl. No.: 18/605,444

(22) Filed: Mar. 14, 2024

Related U.S. Application Data
(63) Continuation of application No. PCT/CN2023/101604, filed on Jun. 21, 2023.
(60) Provisional application No. 63/366,907, filed on Jun. 23, 2022.

(51) Int. Cl. C07F 5/02; A61K 31/69; A61P 31/04

(52) U.S. Cl. CPC C07F 5/025; A61K 31/69; A61P 31/04

(57) ABSTRACT

Described herein are prodrugs of antimicrobial organoboron compounds of Formula I, or salts thereof, and pharmaceutical compositions thereof, methods for their use, and methods for preparing the same.

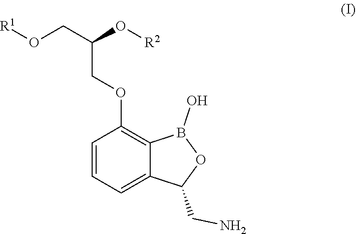

### FIELD

Described herein are prodrugs of antimicrobial boron-organic compounds, pharmaceutical compositions thereof, methods for their use, and methods for preparing the same.

### BACKGROUND

Owing to an increasing bacterial resistance, novel classes of antibacterial compounds are needed for the treatment of microbial infections. These novel agents are required to possess useful activity against key mammalian pathogens, including Gram-negative bacteria, including Pseudomonas aeruginosa, Acinetobacter baumannii, Escherichia coli, and Klebsiella pneumoniae, as well as key Gram-positive bacteria such as multidrug-resistant staphylococci and streptococci. Agents acting via new mechanisms of action are particularly advantageous to avoid undesired cross-resistance with existing drugs.

For many patients with bacterial infections, an oral formulation is the most appropriate choice. Advantages of oral over the intravenous route are the absence of cannula-related infections, a lower drug cost, and a reduction in hidden costs such as the need for a health professional and equipment to administer intravenous antibiotics. Oral therapy is particularly important to improve patient compliance for those who require long-term treatment.

Several antimicrobial boron-organic compounds have been previously described in PCT Applications WO 2008/157726 and WO 2010 080558 and US Application US 2009/0227541. To date, no compound of this class has been approved for anti-infective therapy in human.

A boron-organic compound (shown below) was described in US Application US 2013/0165411.

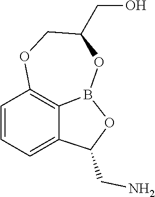 [SMILES: NC[C@H]1OB2O[C@H](CO)COc3cccc1c32]

Compounds of this class are particularly active against Gram-negative bacteria such as Pseudomonas aeruginosa, Acinetobacter baumannii, Escherichia coli, and Klebsiella pneumoniae. Moreover, this type of molecule has good efficacy in a P. aeruginosa neutropenic mouse thigh infection model with subcutaneous administration. However, no oral formulation of this molecule has been reported so far. In fact, many antibiotics, including cephalosporins, can only be given intravenously, A drug with poor oral bioavailability is not suitable for development of an oral drug due to the lack of sufficient exposure of the drug. Poor oral bioavailability can also require much higher dosage, which may induce additional side effects.

There are two main approaches to improve the membrane permeability and thus enhance oral absorbability of a compound. One involves changing the chemical structure, and the other involves developing a formulation without altering the molecular structure. The former can be accomplished by attaching relatively small structure-modifying group, such as an alkyl group or an acyl group to a suitable substituent within the drug, such as a carboxy group or an amino group, to form a prodrug.

Preferred compounds provided herein are stable in prodrug forms before absorption while exhibiting an improved absorption due to a unique prodrug form. Subsequent to administration to a mammal in need of therapy, such prodrug molecules are rapidly converted into active drug chemically and/or enzymatically within bodily compartments, such as intestines, liver, and/or plasma. This desired conversion into an active drug may occur either during and/or after absorption.

However, it is very difficult to develop ideal prodrugs that satisfy all of the aforementioned conditions. For example, prodrugs having an ester bond can be more likely to be hydrolyzed, which may influence chemical stability before absorption. An amide bond may induce a significant change of the physical property, which may in turn negatively influence membrane permeability, such as oral absorbability. Moreover, an amide bond is less likely to be hydrolyzed, which may affect the biotransformation of the compound to an active form and the plasma concentration of the active form. Furthermore, it is difficult to predict the pharmacokinetic profiles of prodrugs because enzymes that control biotransformation of prodrugs to active forms are substrate-specific and particularly, for example, the steric hindrance of a substituent inserted for the formation of a prodrug may prevent reaction of the enzymes. For these reasons, it cannot be predicted how or whether a prodrug will enhance the plasma concentrations of an active form, whether a prodrug will have improved membrane permeability, and/or whether the prodrug will transform to the active form in vivo.

### SUMMARY

Described herein are novel prodrugs of boron compounds with high antibacterial activity against Gram-negative bacteria, such as Pseudomonas aeruginosa, Acinetobacter baumannii, Escherichia coli, and Klebsiella pneumoniae.

It has been found that the prodrug described herein enhance the in vivo exposure of the corresponding active form using animal experiments with the active forms and prodrugs as test drugs.

In one aspect provided herein is a compound of Formula I:

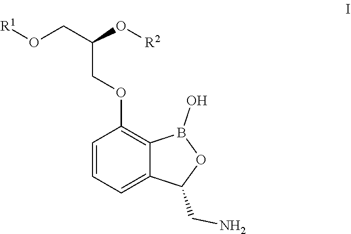

or a pharmaceutically acceptable salt, complex, or tautomer thereof, wherein:
R¹ is selected from H, C₁₋₂₄alkyl-C(═O)—, C₁₋₂₄alkoxy-C(═O)—, C₃₋₇cycloalkyl-C(═O)—, heteroalkyl-C(═O)—, aryl-C(═O)—, heteroaryl-C(═O)—, and (5-methyl-1,3-dioxol-2-one-4-yl)methyl; and
R² is selected from C₁₋₂₄alkyl-C(═O)—, C₁₋₂₄alkoxy-C(═O)—, C₃₋₇cycloalkyl-C(═O)—, heteroalkyl-C(═O)—, aryl-C(═O)—, heteroaryl-C(═O)—, and (5-methyl-1,3-dioxol-2-one-4-yl)methyl; or
R¹ and R² taken together form a heterocyclic group selected from 1,3-dioxane, 2-C₁₋₆-alkyl-1,3-dioxane, 2,2-di(C₁₋₆-alkyl)-1,3-dioxane, 2-methyl-1,3-dioxane, 2-aryl-1,3-dioxane, 2-(2-carboxyphenyl)-1,3-dioxane, 2-(4-carboxyphenyl)-1,3-dioxane, or 2-C₁₋₆-alkylOC(═O)—1,3-dioxane; each of which is optionally substituted with one to four R³;
R³ at each occurrence is independently selected from the group consisting of halo, hydroxy, C₁-C₆ alkyl, C₃-C₆ cycloalkyl, C₁-C₆ alkoxy, aryl, and heteroaryl; or
two R³ groups when attached to adjacent carbons are taken together with the carbons to which they are attached to form a fused C₃-C₆ cycloalkyl; or
two R³ groups when attached to the same carbon are taken together with the carbon to which they are attached to form a spiro C₃-C₆ cycloalkyl;
wherein each R³ is optionally independently substituted with one to three fluoro, hydroxy, or C₁-C₃ alkyl.

In one preferred embodiment of Formula I, R¹ and R² are both alkyl.

In yet another preferred embodiment of Formula I, R¹ and R² are both C₁-C₆ alkyl-C(═O)—.

In another aspect, provided herein is a compound of Formula II:

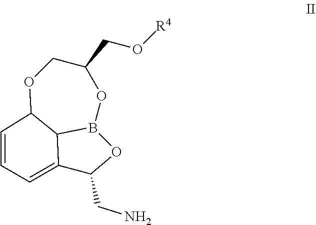

or a pharmaceutically acceptable salt thereof;
wherein:
R⁴ is selected from C₁₋₂₄alkoxy-C(═O)—, C₃₋₇cycloalkyl-C(═O)—, heteroalkyl-C(═O)—, aryl-C(═O)—, heteroaryl-C(═O)—, and (5-methyl-1,3-dioxol-2-one-4-yl)methyl.

In another aspect, provided herein is a pharmaceutical composition comprising a therapeutically effective amount of a compound of Formula I or Formula II, or a pharmaceutically acceptable salt, complex, or tautomer thereof, and a pharmaceutically acceptable carrier.

In another aspect, provided herein is a pharmaceutical composition comprising a compound of Formula I or Formula II, or a pharmaceutically acceptable salt thereof, and a pharmaceutically acceptable carrier.

In an additional aspect, provided herein is a method for the treatment of a microbial infection in a mammal comprising administering to the mammal in need thereof a therapeutically effective amount of a compound of Formula I or Formula II, or a pharmaceutically acceptable salt, complex, or tautomer thereof. The compound of Formula I or Formula II or a pharmaceutically acceptable salt, complex, or tautomer thereof may be administered orally, parenterally, transdermally, topically, rectally, or intranasally in a pharmaceutical composition, including a solution or a powder composition for inhalation.

In an additional aspect, the compound or a pharmaceutically acceptable salt, complex, or tautomer thereof is administered to the mammal orally in a pharmaceutical composition.

In an additional aspect, provided herein is a method for treating mycobacteria microbial infections in humans or other warm-blooded animals by administering to the subject in need thereof a therapeutically effective amount of a compound of Formula I or Formula II or a pharmaceutically acceptable salt thereof. The compound of Formula I or Formula II may be administered orally, parenterally, transdermally, topically, rectally, or intranasally in a pharmaceutical composition.

In another aspect, provided herein are compositions and methods for the treatment of microbial infections caused by microorganisms selected from Gram-negative bacteria, including, but not limited to, Pseudomonas aeruginosa, Acinetobacter baumannii, Escherichia coli, and Klebsiella pneumoniae.

In one embodiment, the method is for the treatment of a skin, soft tissue, respiratory, blood, intra-abdominal, urinary, or eye infection.

In yet another aspect, provided herein are novel intermediates and processes for preparing compounds of Formula I.

### DETAILED DESCRIPTION

Unless otherwise stated, the following terms used in the specification and Claims have the meanings given below:

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₇ alkyl refers to alkyl of one to seven carbon atoms, inclusive.

The terms “alkyl”, “alkenyl”, etc. refer to both straight and branched groups, but reference to an individual radical such as “propyl” embraces only the straight chain radical and a branched chain isomer such as “isopropyl” embraces only the branched chain radical. The alkyl, alkenyl, etc. group may be optionally substituted with one, two, or three substituents selected from the group consisting of halo, aryl, Het¹, or Het². Representative examples include, but are not limited to, difluoromethyl, 2-fluoroethyl, trifluoroethyl. —CH═CH-aryl, —CH═CH-Het¹, —CH₂-phenyl, and the like.

The term “cycloalkyl” means a cyclic saturated monovalent hydrocarbon group of three to six carbon atoms, e.g., cyclopropyl, cyclohexyl, and the like. The cycloalkyl group may be optionally substituted with one, two, or three substituents selected from the group consisting of halo, aryl, Het¹, or Het².

The term “heteroalkyl” means an alkyl or cycloalkyl group, as defined above, having a substituent containing a heteroatom selected from N, O, or S(O)ₙ, where n is an integer from 0 to 2, including, hydroxy (OH), C₁₋₄alkoxy, amino, thio (—SH), and the like. Representative substituents include —NRₐR_{b}, —ORₐ, or —S(O)ₙR_{c}, wherein Rₐ is hydrogen, C₁₋₄alkyl, C₃₋₆cycloalkyl, optionally substituted aryl, optionally substituted heterocyclic, or —COR (where R is C₁₋₄alkyl); R_{b} is hydrogen, C₁₋₄alkyl, —SO₂R (where R is C₁₋₄alkyl or C₁₋₄hydroxyalkyl), —SO₂NRR′ (where R and R′ are independently of each other hydrogen or C₁₋₄alkyl), —CONR′R″ (where R′ and R″ are independently of each other hydrogen or C₁₋₄alkyl); n is an integer from 0 to 2; and R_{c} is hydrogen, C₁₋₄alkyl, C₃₋₆cycloalkyl, optionally substituted aryl, or NRₐR_{b} where Rₐ and R_{b} are as defined above. Representative examples include, but are not limited to 2-methoxyethyl (—CH₂CH₂OCH₃), 2-hydroxyethyl (—CH₂CH₂OH), hydroxymethyl (—CH₂OH), 2-aminoethyl (—CH₂CH₂NH₂), 2-dimethylaminoethyl (—CH₂CH₂NHCH₃), benzyloxymethyl, thiophen-2-ylthiomethyl, and the like.

The term “halo” refers to fluoro (F), chloro (Cl), bromo (Br), or iodo (I).

The term “aryl” refers to phenyl, biphenyl, or naphthyl, optionally substituted with 1 to 3 substituents independently selected from halo, —C₁₋₄alkyl, —OH, —OC₁₋₄alkyl, —S(O)ₙC₁₋₄alkyl wherein n is 0, 1, or 2, —C₁₋₄alkylNH₂, —NHC₁₋₄alkyl, —C(═O)H, or —C═N—OR_{d} wherein R_{d} is hydrogen or —C₁₋₄alkyl.

Het¹ at each occurrence is independently a C-linked 5- or 6-membered heterocyclic ring having 1 to 4 heteroatoms selected from the group consisting of oxygen, nitrogen, and sulfur within the ring. Het at each occurrence is independently a N-linked 5 or 6 membered heterocyclic ring having 1 to 4 nitrogen and optionally having one oxygen or sulfur within the ring.

“Optional” or “optionally” means that the subsequently described event or circumstance may, but need not, occur, and that the description includes instances where the event or circumstance occurs and instances in which it does not. For example, “aryl group optionally mono- or di-substituted with an alkyl group” means that the alkyl may but need not be present, and the description includes situations where the aryl group is mono- or disubstituted with an alkyl group and situations where the aryl group is not substituted with the alkyl group.

Compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the (R)- and (S)-stereochemistry rules of Cahn and Prelog, or by the manner in which the molecule rotates the plane of polarized light and is designated as dextrorotatory or levorotatory (i.e., as (+) or (−)-isomers respectively). A chiral compound can exist as either an individual enantiomer or as a mixture thereof. A mixture containing equal proportions of the enantiomers is called a “racemic mixture.”

The compounds described herein may possess one or more asymmetric centers; such compounds can therefore be produced as individual (R)- or (S)-stereoisomers or as mixtures thereof. Unless indicated otherwise, the description or naming of a particular compound in the specification and Claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry,” 4th edition J. March, John Wiley and Sons, New York, 1992).

A “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and Claims includes both one and more than one such carrier.

A “pharmaceutically acceptable salt” of a compound means a salt that is pharmaceutically acceptable and that possesses the desired pharmacological activity of the parent compound. Such salts include:
(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The term “tautomers” means two or more forms or isomers of an organic compound that could be interconverted into each other via a common chemical reaction called tautomerization, generally analogous to that described by Smith et al., in Advanced Organic Chemistry. 2001, 5th Ed. NY: Wiley Interscience., pp. 1218-1223. The concept of tautomerizations is called tautomerism. The tautomerism may be accompanied by a change from a ring structure to an open structure, as observed, for example, for interconversion between the cyclic pyran form and the open chain form of glucose via formation and breaking of a C—O bond. The degree of tautomerism is often affected by a solvent effect, such as a hydration with water, and the media acidity. A related process concerning cyclic boron compounds may involve formation and breaking of a B—O bond as exemplified below:

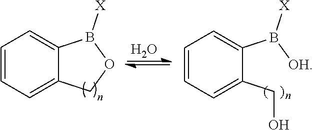

“Treating” or “treatment” of a disease includes:
(1) preventing the disease, i.e. causing the clinical symptoms of the disease not to develop in a mammal that may be exposed to or predisposed to the disease, but does not yet experience or display symptoms of the disease,
(2) inhibiting the disease, i.e., arresting or reducing the development of the disease or its clinical symptoms, or
(3) relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Prodrug” means any compound that releases an active parent drug according to a compound described herein in vivo when such prodrug is administered to a mammalian subject. Prodrugs of a compound of the subject invention are prepared by modifying functional groups present in a compound of the subject invention in such a way that the modifications may be cleaved in vivo to release the parent compound. In certain embodiments, prodrugs include compounds described herein wherein a hydroxy, sulfhydryl, amido or amino group in the compound is bonded to any group that may be cleaved in vivo to regenerate the free hydroxyl, amido, amino, or sulfhydryl group, respectively.

The term “mammal” refers to all mammals including humans, livestock, and companion animals.

“Patient” and “patients” refer to an animal, such as a mammal including a non-primate (e.g., a cow, pig, horse, cat, dog, rat, and mouse) and a primate (e.g., a monkey such as a cynomolgous monkey, a chimpanzee and a human), and for example, a human. In certain embodiments, the patient is a human.

The compounds described herein are generally named according to the IUPAC or CAS nomenclature system. Abbreviations which are well known to one of ordinary skill in the art may be used (e.g. “Ar” for aryl, “Ph” for phenyl, “Me” for methyl, “Et” for ethyl, “h” for hour or hours and “rt” or “r.t.” for room temperature).

### Illustrative Embodiments

Specific and preferred values listed below for radicals, substituents, and ranges, are for illustration only; they do not exclude other defined values or other values within defined ranges for the radicals and substituents.

In some preferred compounds described herein, C₁₋₄alkyl can be methyl, ethyl, propyl, isopropyl, butyl, iso-butyl, sec-butyl, or isomeric forms thereof.

In some preferred compounds described herein, C₃₋₆cycloalkyl can be cyclopropyl, cyclobutyl, cyclopentyl, cyclohexyl, or isomeric forms thereof.

In some preferred compounds described herein, C₁₋₄ heteroalkyl can be hydroxymethyl, hydroxyethyl, or 2-methoxyethyl.

In some preferred compounds described herein, halo can be fluoro (F) or chloro (Cl).

A preferred group of compounds of Formula I includes:

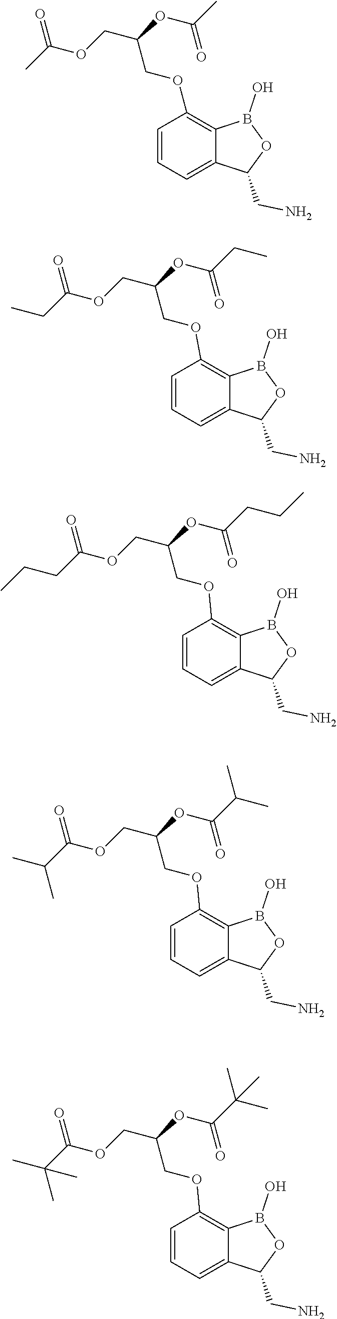

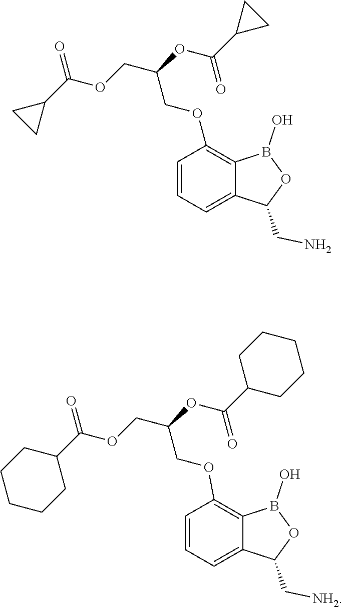 [SMILES: NC[C@H]1OB(O)c2c(OC[C@@H](COC(=O)C3CC3)OC(=O)C3CC3)cccc21 ; NC[C@H]1OB(O)c2c(OC[C@@H](COC(=O)C3CCCCC3)OC(=O)C3CCCCC3)cccc21]

An additional preferred group of compounds of Formula I includes:

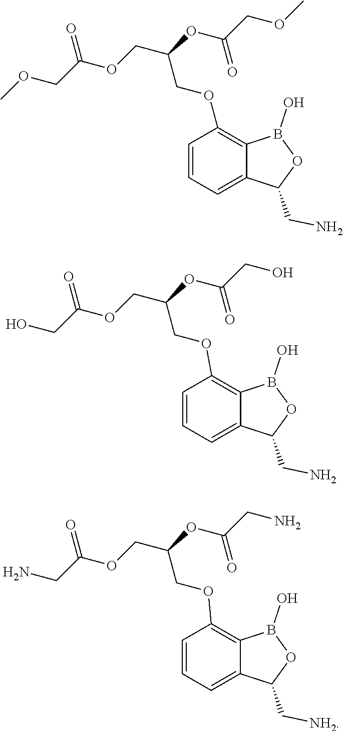 [SMILES: COCC(=O)OC[C@H](COc1cccc2c1B(O)O[C@@H]2CN)OC(=O)COC ; NC[C@H]1OB(O)c2c(OC[C@@H](COC(=O)CO)OC(=O)CO)cccc21 ; NCC(=O)OC[C@H](COc1cccc2c1B(O)O[C@@H]2CN)OC(=O)CN]

An another group of preferred compounds of Formula I includes:

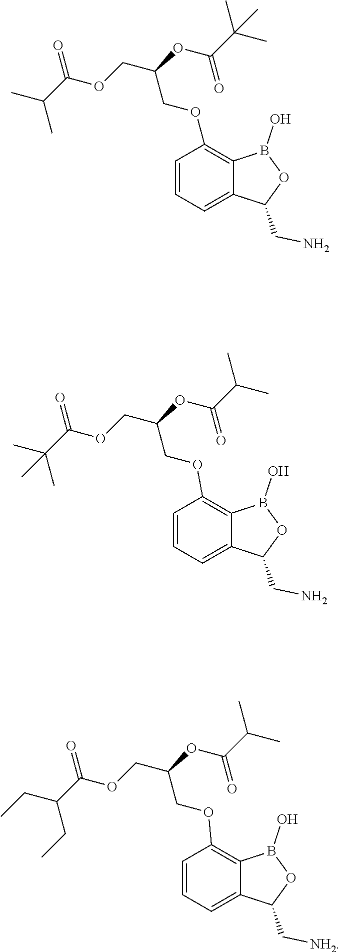 [SMILES: CC(C)C(=O)OC[C@H](COc1cccc2c1B(O)O[C@@H]2CN)OC(=O)C(C)(C)C ; CC(C)C(=O)O[C@H](COC(=O)C(C)(C)C)COc1cccc2c1B(O)O[C@@H]2CN ; CCC(CC)C(=O)OC[C@H](COc1cccc2c1B(O)O[C@@H]2CN)OC(=O)C(C)C]

Yet another group of preferred compounds of Formula I is includes.

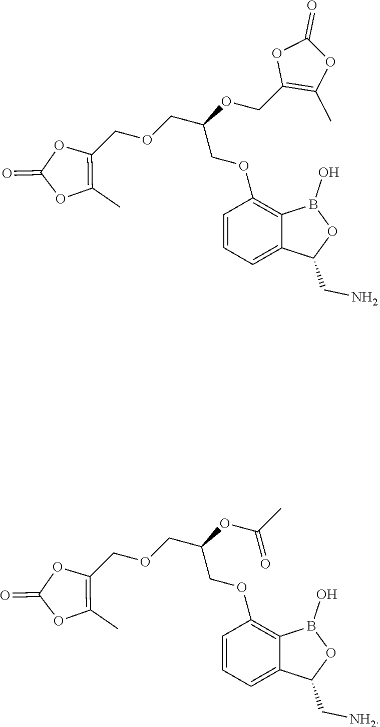 [SMILES: Cc1oc(=O)oc1COC[C@H](COc1cccc2c1B(O)O[C@@H]2CN)OCc1oc(=O)oc1C ; CC(=O)O[C@H](COCc1oc(=O)oc1C)COc1cccc2c1B(O)O[C@@H]2CN]

In some embodiments, the pharmaceutically acceptable salt of preferred compounds is a hydrochloride salt.

### General Synthetic Methods

In certain embodiments, the compounds described herein can be prepared in accordance with one or more of Schemes discussed below. These methods can be used either directly or with obvious variations to a trained chemist to prepare key intermediates and certain compounds described herein.

Additional general methods for preparation of some bicyclic boron compounds are described, for example, in publications US Applications US 2013/0165411 and US 2009/0227541 and PCT Applications WO 2010/080558.

It is also understood that, if so required, any racemic compound(s) or intermediate(s) described herein can be separated into asymmetric chiral materials of a desired optically active isomers using conventional means, including but not limited to chiral liquid chromatography or co-crystallization with a chiral auxiliary reagent, such as a conventional commercial chiral acid or amine.

Suitable synthetic sequences are readily selected per specific structures described herein, but within the art known to individuals practicing organic synthesis, such as methods summarized in available chemistry databases, such as in CAS Scifinder and Elsevier Reaxys. Based on these general methods, the enablement for making the compounds described herein is straightforward and can be practiced within a common professional knowledge. Some general synthetic methods to prepare the compounds described herein are illustrated below in Schemes 1-6 (non-limiting, for illustration only).

One general approach for the synthesis of the compounds of Formula I described herein is illustrated in general Scheme 1.

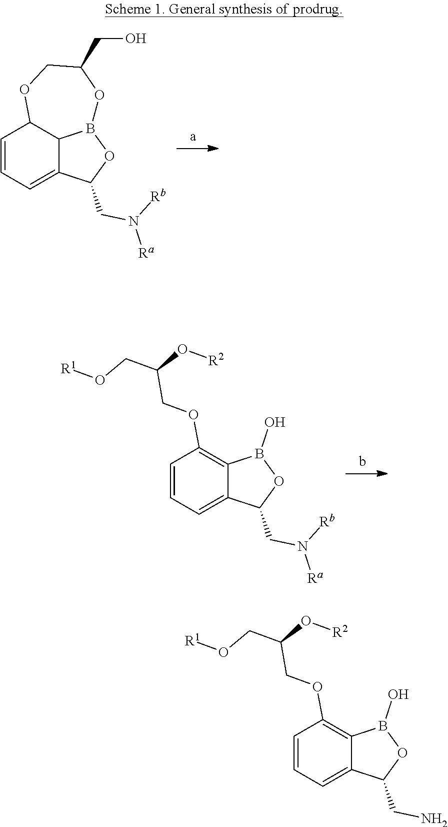

Non-limiting examples of esterification agents for the transformation described in (a) include, but are not limited to, acyl chloride, an anhydride, or an acid with EDC, or the like;

Deprotection agents for the transformation described in (b) depend on the protective group used. For example, in certain embodiments, R^{a} and R^{b} are independently selected from H, Bn, Boc, Fmoc, Cbz and alike.

Additional detailed synthetic schemes for the syntheses of specific compounds described herein are illustrated by methods described for Examples below.

### EXAMPLES

Embodiments are described in the following examples, which are meant to illustrate and not limit the scope described herein. Common abbreviations well known to those with ordinary skills in the synthetic art used throughout. ¹H NMR spectra (δ, ppm) are recorded on 300 MHz instrument in DMSO-d₆ unless specified otherwise. Mass-spectroscopy data for a positive ionization method are provided. Chromatography means silica gel chromatography unless specified otherwise. TLC means thin-layer chromatography. HPLC means reverse-phase HPLC. Unless specified otherwise, all reagents were either from commercial sources, or made by conventional methods described in available literature.

### Example 1

### (2S)-3-acetoxy-1-{[(3S)-3-(aminomethyl)-1-hydroxy-1,3-dihydrobenzo[2,1-c][1,2]oxaborol-7-yl]oxy}propan-2-yl acetate hydrochloride

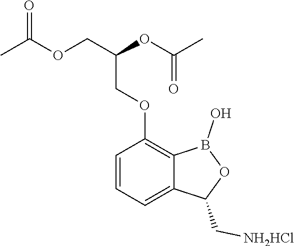

Scheme for preparation of the compound of Example 1:

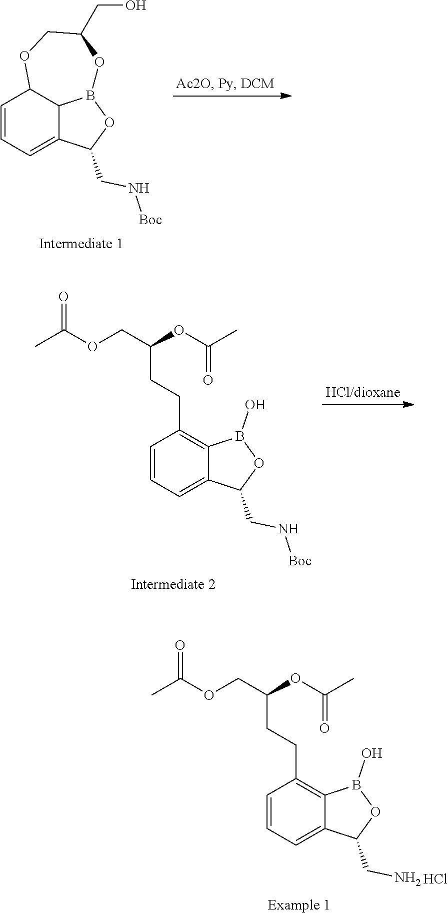

Intermediate 2. To the solution of Intermediate 1 (60 mg, 0.26 mmol, prepared as described in US Application US 2013/0165411) and pyridine. (31 μL, 0.33 mmol) in DCM (2 mL) was added dropwise Ac₂O (32 μL, 0.33 mmol), and the mixture was stirred for 2 hours. After completion of the reaction, the solvent was removed by concentration, and the residue was purified on pre-HPLC to give Intermediate 2 (25 mg). MS (m/z): 438 [M+H].

Example 1. Intermediate 2 was dissolved in dioxane solution of 5 M HCl (2 mL) at room temperature, and the mixture was stirred for 1 hour. Thereafter, the mixture was lyophilized to afford the compound of Example 1 (16.9 mg) as light-yellow powder. MS (m/z): 338 [M+H]. ¹H NMR: (400 MHz, D₂O): 7.48 (t, J=8.0 Hz, 1H); 7.01 (d, J=7.6 Hz, 1H); 6.92 (dd, J=12.0, 8.0 Hz, 1H); 5.35 (dd, J=7.4, 3.0 Hz, 1H); 4.35˜4.17 (m, 5H); 3.73˜3.60 (m, 1H); 3.56˜3.51 (m, 2H); 3.08˜3.01(m, 1H); 1.99 (s, 3H), 1.98 (s, 3H).

The compounds below were synthesized following procedures described for Example 2.

| Example | Structure/Name | Chemical Name | [M + H]⁺ | ¹H NMR |
|---|---|---|---|---|
| 2 | 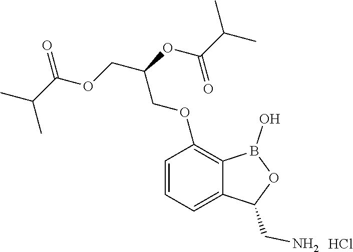 [SMILES: CC(C)C(=O)OC[C@H](COc1cccc2c1B(O)O[C@@H]2CN)OC(=O)C(C)C ; Cl] | (2S)-1-{[(3S)-3- (aminomethyl)-1- hydroxy-1,3- dihydrobenzo[2,1-c] [1,2]oxaborol-7-yl] oxy} -3-[(2- methylpropanoyl) oxy]propan-2-yl 2-methylpropanoate hydrochloride | 394 | ¹H NMR (400 MHZ, MeOD, ppm): δ 7.17 (t, J = 7.7 Hz, 1H), 6.79 (d, J = 7.4 Hz, 1H), 6.71 (d, J = 8.0 Hz, 1H), 5.38 (dd, J = 6.3, 3.9 Hz, 1H), 5.04 (s, 1H), 4.53 (dd, J = 11.9, 3.4 Hz, 1H), 4.38 (dd, J = 11.9, 6.8 Hz, 1H), 4.19 (t, J = 4.0 Hz, 2H), 3.31 (s, 2H), 3.24- 3.18 (m, 1H), 3.04 (dd, J = 12.5, 3.9 Hz, 1H), 2.55 (dt, J = 13.2, 6.4 Hz, 2H), 1.15 (t, J = 6.8 Hz, 12H) |
| 3 | 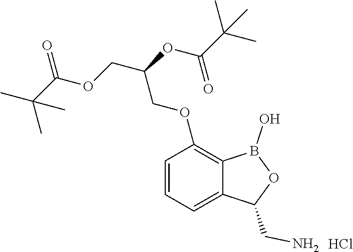 [SMILES: CC(C)(C)C(=O)OC[C@H](COc1cccc2c1B(O)O[C@@H]2CN)OC(=O)C(C)(C)C ; Cl] | (2S)-3-[(2,2- dimethylpropanoyl) oxy]-1-{[(3S)-3- (aminomethyl)-1- hydroxy-1,3- dihydrobenzo[2,1-c] [1,2]oxaborol-7-yl] oxy}propan-2-yl 2,2- dimethylpropanoate hydrochloride | 422 | ¹H NMR (300 MHz, DMSO-d₆, ppm): δ 8.63 (s, 1H), 7.40 (t, J = 7.8 Hz, 1H), 7.01 (d, J = 7.5 Hz, 1H), 6.85 (d, J = 8.1 Hz, 1H), 5.30 (s, 1H), 4.98 (dd, J = 6.6, 3.6 Hz, 1H), 4.43 (dd, J = 12.0, 3.1 Hz, 1H), 4.35-4.18 (m, 3H), 2.96 (dd, J = 13.2, 3.9 Hz, 1H), 2.62 (dd, J = 13.3, 6.8 Hz, 1H), 1.12 (s, 9H), 1.09 (s, 9H) |
| 6 | 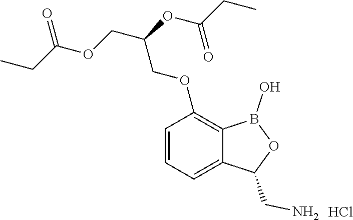 [SMILES: CCC(=O)OC[C@H](COc1cccc2c1B(O)O[C@@H]2CN)OC(=O)CC ; Cl] | (2S)-1-{[(3S)-3- (aminomethyl)-1- hydroxy-1,3- dihydrobenzo[2,1-c] [1,2]oxaborol-7- yl]oxy}-3-[(2- methylpropanoyl) oxy]propan-2-yl propanoate hydrochloride | 366 | ¹H NMR (400 MHZ, D₂O, ppm): 7.24 (d, J = 7.2 Hz, 1H); 6.86-6.78 (m, 2H); 5.40 (s, 1H); 5.10 (s, 1H); 4.43-4.19 (m, 4H); 3.33-3.30 (m, 1H); 3.12-3.08 (m, 1H); 2.35-2.68 (m, 4H); 1.01- 0.97 (m, 6H) |

### Example 4

### [(2S,6S)-2-(aminomethyl)-4-bora-3,5,8-trioxatricyclo[7.3.1.04,13]trideca-1(12),9(13),10-trien-6-yl]methyl 2-methylpropanoate hydrogen chloride

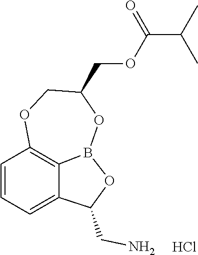 [SMILES: CC(C)C(=O)OC[C@@H]1COc2cccc3c2B(O1)O[C@@H]3CN ; Cl]

The compound of Example 4 was prepared according to the methods described in US Application US 2013/0165411.

### Example 5

### [(2S,6R)-2-(aminomethyl)-4-bora-3,5,8-trioxatricyclo[7.3.1.04,13]trideca-1(12),10-dien-6-yl]methanol hydrochloride

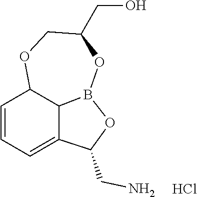

The compound of Example 5 was prepared according to the methods described in US Application US 2013/0165411.

### Example 7

### [(2S,6S)-2-(aminomethyl)-4-bora-3,5,8-trioxatricyclo[7.3.1.04,13]trideca-1(12),9(13),10-trien-6-yl]methyl acetate hydrogen chloride

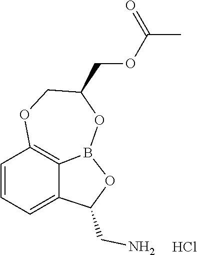 [SMILES: CC(=O)OC[C@@H]1COc2cccc3c2B(O1)O[C@@H]3CN ; Cl]

The compound of Example 7 was prepared according to the methods described in US Application US 2013/0165411.

### Example 8

### [(2S,6S)-2-(aminomethyl)-4-bora-3,5,8-trioxatricyclo[7.3.1.04,13]trideca-1(12),9(13),10-trien-6-yl]methyl propanoate hydrogen chloride

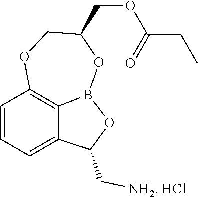

The compound of Example 8 was prepared according to the methods described in US Application US 2013/0165411.

### Utility and Testing

The compounds described herein are prodrugs, which are expected to convert to the parent boron compound to exert antibacterial efficacy. The antibacterial activity of the parent boron compound has been disclosed in US Application US 2013/0165411. Thus, the compounds described herein are useful antimicrobial agents and may be effective against a number of human and veterinary pathogens, including Pseudomonas aeruginosa, Acinetobacter baumannii, Escherichia coli, and Klebsiella pneumoniae.

The in vitro activity of compounds described herein may be assessed by standard testing procedures such as the determination of minimum inhibitory concentration (MIC) as described in Approved Standard. Methods for Dilution Antimicrobial Susceptibility Tests for Bacteria that Grow Aerobically, 3^{rd} ed., 1993, published by the National Committee for Clinical Laboratory standards, Villanova, Pennsylvania, USA. Low MIC values indicate high antibacterial activity, while high MIC values reveal a reduced antibacterial activity (with higher drug concentration required for pathogen eradication in the latter instance). Generally, MIC values of about ≤4-8 μg/mL indicate a therapeutic (i.e. suitable for therapy) potency for antibacterial drugs, while MIC values of ≥16 μg/mL indicate a lack of therapeutically useful activity for a test compound.

The useful in vitro activity (potency) of representative compounds described herein against mycobacteria such as Pseudomonas aeruginosa, Acinetobacter baumannii, Escherichia coli, and Klebsiella pneumoniae. is illustrated by the MIC data of Table 1 below.

As evident from the data of Table 1, the reference compound of Example 5 is highly active against many Gram-negative bacteria, including Pseudomonas aeruginosa, Acinetobacter baumannii, Escherichia coli, and Klebsiella pneumoniae (MIC ranges from 2-4 μg/mL). Typical for prodrug derivatives, the prodrugs of Example 5, such as the compounds of Example 2, 3, 4, and 8 are considered inactive.

TABLE 1
In vitro antibacterial activity of representative Examples.

| | P. | A. baumannii | K. | E. coli |
|---|---|---|---|---|
| | aeruginosa | MABA | pneumoniae | MECO |
| | PAE 1001 | 0001 | ATCC10031 | 002 |
| | μg/mL | μg/mL | μg/mL | μg/mL |
| Reference | 2.0 | 2.0 | 4.0 | 2.0 |
| Example 5 | | | | |
| Example 1 | >32 | >32 | >32 | >32 |
| Example 2 | >32 | >32 | >32 | >32 |
| Example 3 | >32 | >32 | >32 | >32 |
| Example 4 | >32 | >32 | >32 | >32 |
| Example 8 | >32 | >32 | >32 | >32 |

In addition to in vitro activity (potency determined as MIC), in vivo efficacy or ability to eradicate bacterial pathogens to the effect of survival of mammals under therapy is critical. It is well established that compounds with similar antibacterial potency in vitro (MICs) may display dramatically different activity in vivo. This can result in a desired therapeutic effect for some efficacious compounds, or a lack of any useful anti-infective effect for other, non-efficacious compounds. This is critical for the actual therapy outcome, and is determined by multiple factors affecting the compound behavior in vivo, such as its absorption, distribution, metabolism, and excretion.

Furthermore, in vivo activity (or efficacy) is generally the activity most critical for prodrug compounds that are generally inactive in vitro, and release an active drug after administration to a mammal in the need of a therapy.

To establish the efficacy of the compounds described herein in vivo, a Pseudomonas aeruginosa mouse lung infection model was performed by administering test compounds analogously to the method described in Andes et al. in Antimicrobial Agents and Chemotherapy, 2002, 46(11), 3484-3489. In this model, a greater reduction in the bacterial colony-forming units (CFU) indicates more beneficial therapeutic effect (more bacterial eradication), while a lower CFU reduction indicates a lower effect (less bacterial eradication). The in vivo antibacterial effect is also referred to as “efficacy”, in contrast to the term “potency,” which is commonly used for in vitro activity (expressed as MIC).

In the Pseudomonas aeruginosa ICR mouse (The mice were randomly divided into groups with 6 mice in each group) lung infection, reference Example 5 was administered orally at 10 mg/kg once daily. Compared to the untreated control group, reference compound of Example 5 displayed weak antibacterial potency and resulted in a 0.53 log reduction of CFU in lung. As a prodrug for oral administration, the compound of Example 2 and 8 were also given by oral gavage at 10 mg/kg once daily. Even though the prodrug itself does not possess antibacterial potency, it displayed good in vivo efficacy. Surprisingly, the efficacy of the compound of Example 2 and 8 when administered at 10 mg/kg is much better than the efficacy of the reference compound of Example 5, with 1.77 and 0.82 log reduction of CFU in lung. The data is strongly supportive of the prodrug of Example 2 and 8 being converted to reference compound of Example 5 after oral administration to exert the antibacterial activity. The data also supports a high conversion rate.

TABLE 2
Data from Pseudomonas aeruginosa mouse lung infection model.

| | | | WPAE 1004, Lg (CFU/lung) | |
|---|---|---|---|---|
| | | Dose, | | Δ 24 h |
| Cpds | Route | mg/kg | 24 h | (Control-Theray) |
| Control | — | 0 | 9.27 ± 0.32 | 0 |
| Example 2 | po | 10 | 7.50 ± 0.73 | 1.77 |
| Example 8 | po | 10 | 8.45 ± 0.64 | 0.82 |
| Control | — | 0 | 9.09 ± 0.32 | 0 |
| Example 5 | po | 10 | 8.56 ± 0.49 | 0.53 |

po, oral administration, oral suspension used herein.

To further elucidate therapeutic potential of drug compounds, pharmacokinetic (PK) data is used to establish the key parameters predictive of the therapy outcome, such as area under the curve (AUC) for a plot monitoring the change in the systemic drug concentration over time. Thus, a higher AUC value indicates a greater exposure to the drug, commonly associated with a greater therapeutic potential due to a higher amount of drug available to combat the infection in a mammal. In contrast, a lower AUC value indicates a reduced exposure to the drug under study, resulting in a reduced amount of antibiotic available to combat bacterial infestations. To that end, the compounds described herein were tested in a rat PK model of oral administration performed analogously to methods described in the monograph Current Protocols in Pharmacology, 2005, 7.1.1-7.1.26, John Wiley & Sons, Inc.

All the compounds were administered to SD rats (The rats were randomly divided into groups with 3 rats in each group) by intravenous (iv) or oral gavage. Since the prodrugs are expected to convert to the parent molecule in vivo, only the parent compound (the compound of Example 5) was tested for all samples. As shown in Table 3, the parent compound of Example 5 only had a low oral bioavailability of 15%. This low bioavailability as well as low exposure (AUC) and C max make it inappropriate for development as oral drug. Quite surprisingly, the pharmacokinetic data for the compounds of this invention revealed a greatly improved systemic exposure (AUC) and C max at the same dosage of 5 mg/kg. The AUC of Examples 1, 2, 3, 4, 6, 7 and 8 are all significantly higher than that of Example 5, in spite of the molecular weight of these prodrugs are higher than Example 5. For example, the compound of Example 2 displayed AUC and C max as 2906 ng*h/mL and 870 ng/ml, respectively. This unexpected result represents a striking 3.4-fold and 3-fold improvement in the exposure and C max, respectively, which is line with the improved efficacy described in Table 2. Because the prodrugs usually have larger molecular weight due to the introduction of prodrug substructures, the AUC is corrected by dose and molecular weight (MW) to give AUC per mole, to compare the efficiency among the prodrugs. Importantly, the compound of Example 2 also displayed significantly higher AUC per mole compared to the compound of Example 4, which was previously described in US 2013/0165411.

TABLE 3
Pharmacokinetic study of selected Examples in rat.

| | | Administration | Dosage | AUC | Cₘₐₓ, | Oral | AUC per |
|---|---|---|---|---|---|---|---|
| Compound | Structure | Route | mg/kg | ng*h/ml | ng/ml | bioavailability | mole* |
| Example 5 | 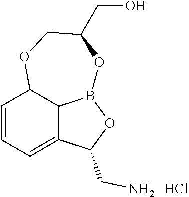 | iv oral | 5 5 | 5661 856 | 6776 139 | / 15% | 307.4 46.5 |
| Example 1 | 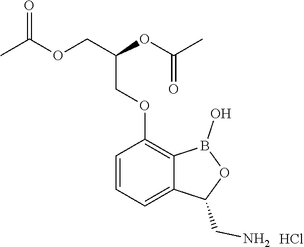 [SMILES: CC(=O)OC[C@H](COc1cccc2c1B(O)O[C@@H]2CN)OC(C)=O ; Cl] | oral | 5 | 1251 | 241 | 22% | 100.5 |
| Example 2 | 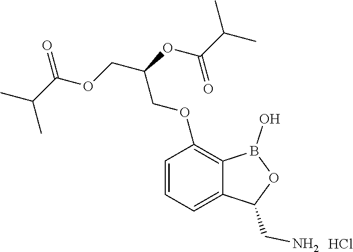 [SMILES: CC(C)C(=O)OC[C@H](COc1cccc2c1B(O)O[C@@H]2CN)OC(=O)C(C)C ; Cl] | oral | 5 | 2906 | 870 | 51% | 249.5 |
| Example 4 | 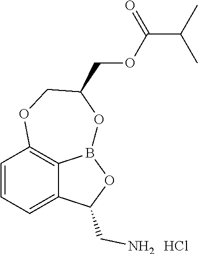 [SMILES: CC(C)C(=O)OC[C@@H]1COc2cccc3c2B(O1)O[C@@H]3CN ; Cl] | oral | 5 | 2232 | 581 | 39% | 152.5 |
| Example 6 | 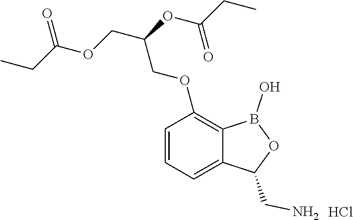 [SMILES: CCC(=O)OC[C@H](COc1cccc2c1B(O)O[C@@H]2CN)OC(=O)CC ; Cl] | oral | 10 | 3113 | / | 27% | 125.0 |
| Example 7 | 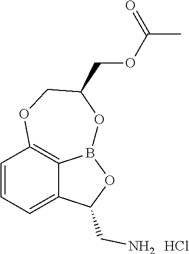 [SMILES: CC(=O)OC[C@@H]1COc2cccc3c2B(O1)O[C@@H]3CN ; Cl] | oral | 10 | 3944 | / | 30% | 123.6 |
| Example 3 | 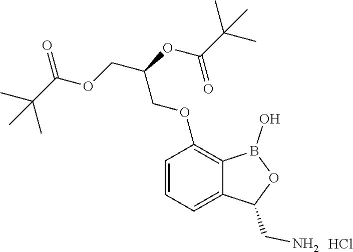 [SMILES: CC(C)(C)C(=O)OC[C@H](COc1cccc2c1B(O)O[C@@H]2CN)OC(=O)C(C)(C)C ; Cl] | oral | 10 | 3279 | / | 29% | 150.1 |
| Example 8 | 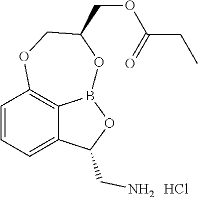 [SMILES: CCC(=O)OC[C@@H]1COc2cccc3c2B(O1)O[C@@H]3CN ; Cl] | oral | 10 | 5421 | / | 38% | 177.6 |

$* AUC per mole is AUC corrected by dose and molecular weight , = \frac{AUC}{Dose / MW of Example X}$

Moreover, in a lung distribution study conducted in Balb/C mouse (three mice per time point), Example 2 showed much higher exposure in the lung than in the plasma (assessed by the area under the lung/plasma concentration time curve (AUC). As shown in Table 4, compounds of Example 5 and 2 were administered at 10 mg/kg via iv and oral, respectively. In analysis of Example 2, the concentration of parent compound (Example 5) and prodrug (Example 2) were determined in both plasma and lung. The prodrug (Example 2) were rapidly converted to Example 5, with little prodrug can be detected in plasma. The oral bioavailability of Example 5 generated from Example 2 is 83.95% in mice, compared to the AUC of Example 5 administered by iv. Despite the rapid conversion of the prodrug, it is very surprised to see more Example 5 were detected in lung, with a lung/plasma AUC ratio of 5.24, which is almost 2.2-fold higher than that of Example 5 administered by iv. The higher accumulation of drugs in lung is particularly useful for treatment of pneumonia, which is also in agreement with the superior efficacy of Example 2 in Pseudomonas aeruginosa mouse lung infection model (Table 2).

TABLE 4
Lung vs. plasma distribution of the drug of Example
5 and its prodrug of Example 2 in mice.

| | | | Example 2 | | | |
|---|---|---|---|---|---|---|
| | Example 5 | | 10 mg/kg, po | | | |
| | 10 mg/kg, iv | | Levels for | | Levels for | |
| | Example 5 | | Example 5 | | Example 2 | |
| | plasma | lung | plasma | lung | plasma | lung |
| t_{1/2}, h | 1.87 | 1.46 | 1.93 | 2.09 | NA | 6.42 |
| AUCₗₐₛₜ | 5359 | 12822 | 4499 | 23589 | 0.0421 | 968 |
| | ng*h/ml | ng*h/g | ng*h/ml | ng*h/g | ng*h/ml | ng*h/g |

| AUC ratio | 2.39 | | 5.24 | / | |
|---|---|---|---|---|---|
| (lung/plasma) | | | | | |
| Oral | / | / | 83.95% | / | / |
| bioavailability | | | | | |

This dramatic improvement of in vivo exposure (AUC) after oral administration for the compound of Example 2 in both plasma and lung is entirely unexpected and very surprising. Additional related compounds provided herein also display surprisingly improved in vivo exposure. Thus, pharmacokinetic data for the compound of Example 2 in a rat model of oral administration reveals a greatly improved systemic exposure and Cₘₐₓ over the parent compound of Example 5.

Above representative data taken in its entirety reveal a surprisingly superior therapeutic potential for the compounds described herein, with the beneficial unexpected advantages in areas of potency, efficacy, and exposure. The dramatic and surprising improvement in three distinctly different critical parameters for antibacterial compounds provided herein offers marked benefits for human or mammal therapy, including but not limited, to convenient oral administration for long therapy duration, a reduced effective drug dose, and reduced possible adverse effects.

### Administration and Pharmaceutical Formulations

In general, the compounds described herein are administered in a therapeutically effective amount by any of the accepted modes of administration for agents that serve similar utilities. By way of example, the compounds described herein may be administered orally, parenterally, transdermally, topically, rectally, or intranasally. The actual amount of a compound described herein, i.e., the active ingredient, will depend on a number of factors, such as the severity of the disease, i.e., the infection, to be treated, the age and relative health of the subject, the potency of the compound used, the route and form of administration, and other factors, all of which are within the purview of the attending clinician.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method described herein, the therapeutically effective dose can be estimated initially from cell culture assays. A dose may be formulated in animal models to achieve a circulating plasma concentration range which includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

When employed as pharmaceuticals, the compounds described herein are usually administered in the form of pharmaceutical compositions. These compounds can be administered by a variety of routes including oral, parenteral, transdermal, topical, rectal, and intranasal.

These compounds are effective as both injectable and oral compositions. Such compositions can be prepared in a manner well known in the pharmaceutical art and comprise at least one active compound.

Also described herein are pharmaceutical compositions which contain, as the active ingredient, one or more of the compounds described herein with a pharmaceutically acceptable carrier. In making the compositions described herein, the active ingredient is usually mixed with an excipient, diluted by an excipient or enclosed within such a carrier, which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions described herein can be formulated so as to provide quick, sustained or delayed release of the active ingredient after administration to the patient by employing procedures known in the art.

The quantity of an active component, that is the compound described herein, in the pharmaceutical composition and unit dosage form thereof may be varied or adjusted widely depending upon the particular application, the potency of the particular compound and the desired concentration.

The compositions are preferably formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Preferably, a compound described herein is employed at no more than about 20 weight percent of the pharmaceutical composition, more preferably no more than about 15 weight percent, with the balance being pharmaceutically inert carrier(s).

The active compound is effective over a wide dosage range and is generally administered in a pharmaceutically or therapeutically effective amount. It, will be understood, however, that the amount of the compound actually administered will be determined by a physician, in light of relevant circumstances, including, but not included to the condition to be treated, the severity of the bacterial infection being treated, the chosen route of administration, the actual compound administered, the age, weight, and response of the individual patient, the severity of the patient's symptoms, and the like.

In a therapeutic use for treating, or combating, bacterial infections in warm-blooded animals, the compounds or pharmaceutical compositions thereof can be administered orally, topically, transdermally, and/or parenterally at a dosage to obtain and maintain a concentration, that is, an amount, or blood-level of active component in the animal undergoing treatment which will be antibacterially effective. Generally, such antibacterially or therapeutically effective amount of dosage of active component (i.e., an effective dosage) will be in the range of about 0.1 to about 100, more preferably about 1.0 to about 50 mg kg of body weight/day.

For preparing solid compositions such as tablets, the principal active ingredient is mixed with a pharmaceutical excipient to form a solid preformulation composition containing a homogeneous mixture of a compound described herein. When referring to these preformulation compositions as homogeneous, it is meant that the active ingredient is dispersed evenly throughout the composition so that the composition may be readily subdivided into equally effective unit dosage forms such as tablets, pills and capsules. The solid preformulation is then subdivided into unit dosage forms of the type described above containing from, for example, 0.1 to about 500 mg of the active ingredient described herein.

The tablets or pills described herein may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such as materials including a number of polymeric acids and mixtures of polymeric acids with such materials as shellac, cetyl alcohol, and cellulose acetate.

The liquid forms in which the compositions described herein may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure-breathing machine. Solution, suspension, or powder compositions may be administered, preferably orally or nasally, from devices that deliver the formulation in an appropriate manner.

Another preferred formulation employed in the methods described herein employs transdermal delivery devices (“patches”). Such transdermal patches may be used to provide continuous or discontinuous infusion of a compound described herein in a controlled amount. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 which is herein incorporated by reference.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions that can transiently open the blood-brain barrier.

Other suitable formulations for use of the compounds described herein can be found in Remington's Pharmaceutical Sciences, Mace Publishing Company, Philadelphia, PA, 17th ed. (1985).

As noted above, the compounds described herein are suitable for use in a variety of drug delivery systems described above. Additionally, in order to enhance the in vivo serum half-life of the administered compound, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka, et al., U.S. Pat. Nos. 4,235,871, 4,501,728 and 4,837,028 each of which is incorporated herein by reference.

As noted above, the compounds administered to a patient are in the form of pharmaceutical compositions described above. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the compound preparations typically will be between 3 and 11, more preferably from 5 to 9 and most preferably from 7 and 8. It will be understood that use of certain of the foregoing excipients, carriers, or stabilizers will result in the formation of pharmaceutical salts.

The disclosures of each and every patent, patent application and publication (for example, journals, articles and/or textbooks) cited herein are hereby incorporated by reference in their entirety. Also, as used herein and in the appended claims, singular articles such as “a”, “an” and “one” are intended to refer to singular or plural. While embodiments have been described herein in conjunction with a preferred aspect, a person with ordinary skills in the art, after reading the foregoing specification, can affect changes, substitutions of equivalents and other types of alterations to the embodiments as set forth herein. Each aspect described above can also have included or incorporated therewith such variations or aspects as disclosed in regard to any or all of the other aspects. The description herein is also not to be limited in terms of the particular aspects described herein, which are intended as single illustrations of individual aspects provided herein. Many modifications and variations described herein can be made without departing from its spirit and scope, as will be apparent to those skilled in the art. Functionally equivalent methods within the scope of this description, in addition to those enumerated herein, will be apparent to those skilled in the art from the foregoing descriptions. It is to be understood that this description is not limited to particular methods, reagents, process conditions, materials and so forth, which can, of course, vary. It is also to be understood that the terminology used herein is for the purpose of describing particular aspects only, and is not intended to be limiting. Thus, it is intended that the specification be considered as exemplary.

1. A compound of Formula I: 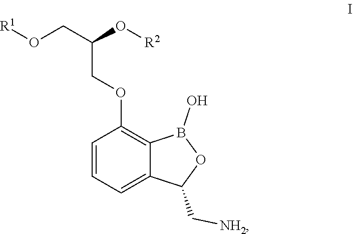
or a pharmaceutically acceptable salt, complex, or tautomer thereof;
wherein:
R¹ is selected from H, C₁₋₂₄alkyl-C(═O)—, C₁₋₂₄alkoxy-C(═O)—, C₃₋₇cycloalkyl-C(═O)—, heteroalkyl-C(═O)—, aryl-C(═O)—, heteroaryl-C(═O)—, and (5-methyl-1,3-dioxol-2-one-4-yl)methyl; and
R² is selected from C₁₋₂₄alkyl-C(═O)—, C₁₋₂₄alkoxy-C(═O)—, C₃₋₇cycloalkyl-C(═O)—, heteroalkyl-C(═O)—, aryl-C(═O)—, heteroaryl-C(═O)—, and (5-methyl-1,3-dioxol-2-one-4-yl)methyl; or
R¹ and R² taken together form a heterocyclic group selected from 1,3-dioxane, 2-C₁₋₆-alkyl-1,3-dioxane, 2,2-di(C₁₋₆-alkyl)-1,3-dioxane, 2-methyl-1,3-dioxane, 2-aryl-1,3-dioxane, 2-(2-carboxyphenyl)-1,3-dioxane, 2-(4-carboxyphenyl)-1,3-dioxane, and 2-C₁₋₆-alkylOC(═O)-1,3-dioxane; each of which is optionally substituted with one to four R³;
R³ at each occurrence is independently selected from the group consisting of halo, hydroxy, C₁-C₆ alkyl, C₃-C₆ cycloalkyl, C₁-C₆ alkoxy, aryl, and heteroaryl; or
two R³ groups when attached to adjacent carbons and taken together with the carbons to which they are attached are a fused C₃-C₆ cycloalkyl; or
two R³ groups when attached to the same carbon and taken together with the carbon to which they are attached are a spiro C₃-C₆ cycloalkyl; wherein each R³ is optionally independently substituted with one to three fluoro, hydroxy, or C₁-C₃ alkyl.

2. The compound of claim 1 selected from: 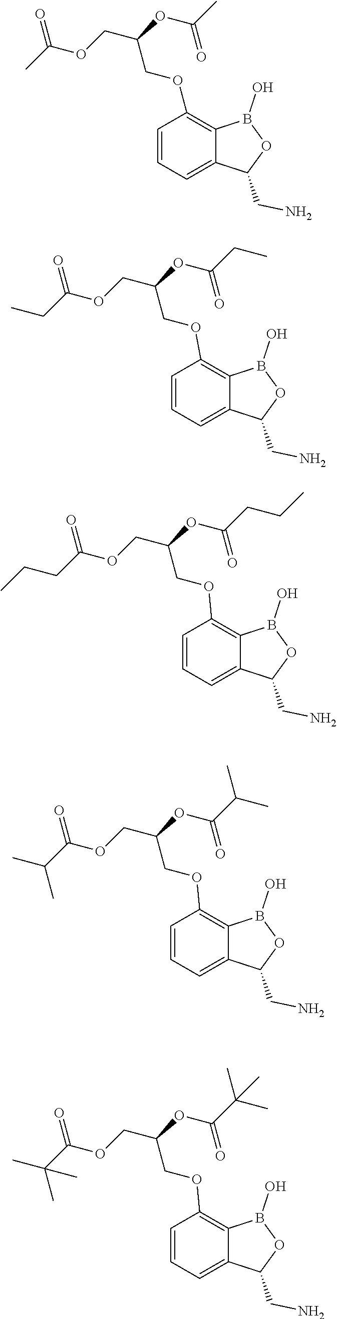 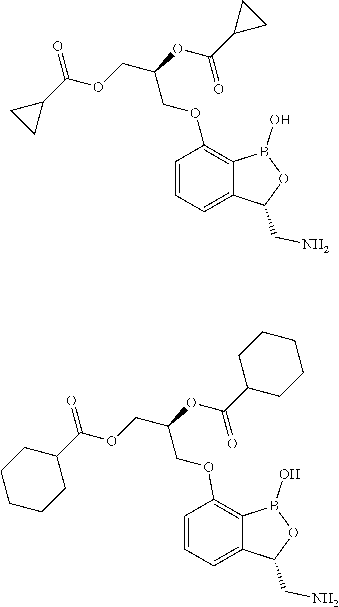 [SMILES: NC[C@H]1OB(O)c2c(OC[C@@H](COC(=O)C3CC3)OC(=O)C3CC3)cccc21 ; NC[C@H]1OB(O)c2c(OC[C@@H](COC(=O)C3CCCCC3)OC(=O)C3CCCCC3)cccc21]
or a pharmaceutically acceptable salt, complex, or tautomer thereof.

3. The compound of claim 1 selected from: 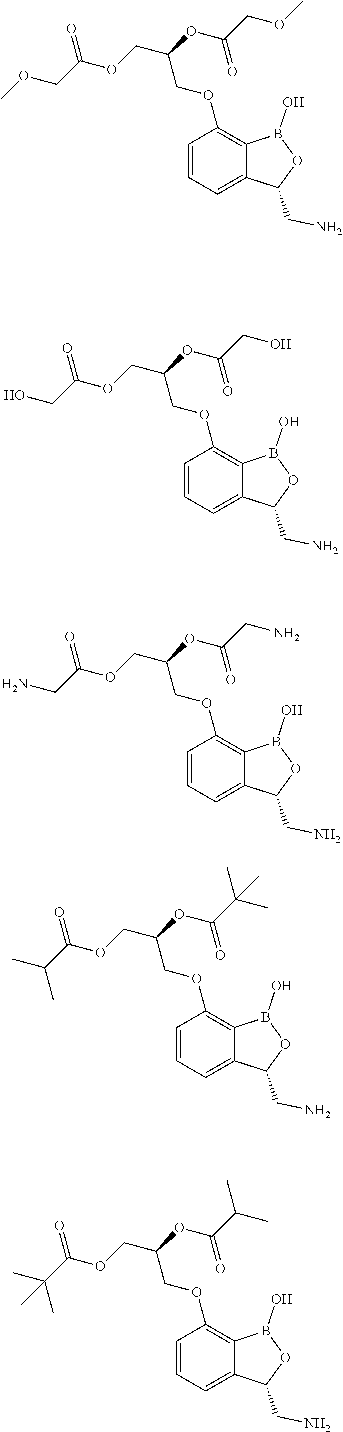 [SMILES: COCC(=O)OC[C@H](COc1cccc2c1B(O)O[C@@H]2CN)OC(=O)COC ; NC[C@H]1OB(O)c2c(OC[C@@H](COC(=O)CO)OC(=O)CO)cccc21 ; NCC(=O)OC[C@H](COc1cccc2c1B(O)O[C@@H]2CN)OC(=O)CN ; CC(C)C(=O)OC[C@H](COc1cccc2c1B(O)O[C@@H]2CN)OC(=O)C(C)(C)C ; CC(C)C(=O)O[C@H](COC(=O)C(C)(C)C)COc1cccc2c1B(O)O[C@@H]2CN] 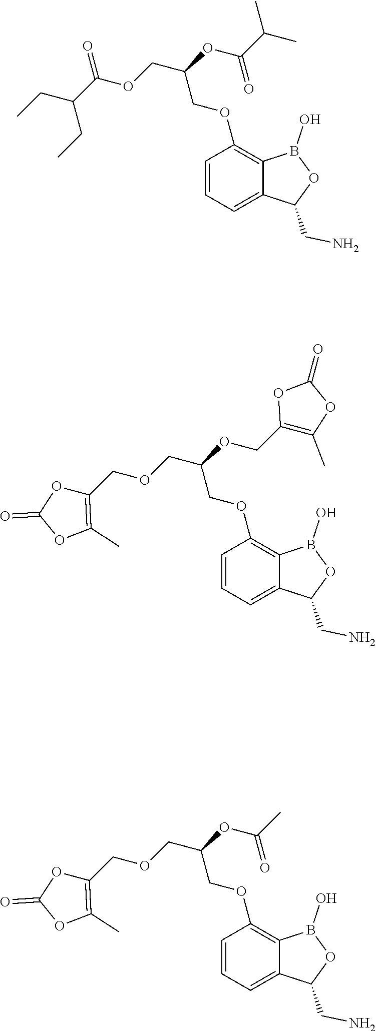 [SMILES: CCC(CC)C(=O)OC[C@H](COc1cccc2c1B(O)O[C@@H]2CN)OC(=O)C(C)C ; Cc1oc(=O)oc1COC[C@H](COc1cccc2c1B(O)O[C@@H]2CN)OCc1oc(=O)oc1C ; CC(=O)O[C@H](COCc1oc(=O)oc1C)COc1cccc2c1B(O)O[C@@H]2CN]
or a pharmaceutically acceptable salt, complex, or tautomer thereof.

4. The compound according to claim 1, or a pharmaceutically acceptable salt, complex, or tautomer thereof, wherein the pharmaceutically acceptable salt is a hydrochloride salt.

5. A method for the treatment of a microbial infection in a mammal comprising administering to the mammal in need thereof a therapeutically effective amount of a compound of claim 1, or a pharmaceutically acceptable salt, complex, or tautomer thereof.

6. The method according to claim 5, wherein the compound or a pharmaceutically acceptable salt, complex, or tautomer thereof is administered to the mammal orally, parenterally, transdermally, topically, rectally, or intranasally in a pharmaceutical composition.

7. The method according to claim 6, wherein the compound or a pharmaceutically acceptable salt, complex, or tautomer thereof is administered to the mammal orally in a pharmaceutical composition.

8. The method according to claim 5, wherein the microbial infection is caused by a Gram-negative bacteria selected from Pseudomonas aeruginosa, Acinetobacter baumannii, Escherichia coli, and Klebsiella pneumoniae.

9. The method according to claim 5, wherein the infection is a skin, soft tissue, respiratory, blood, intraabdominal, urinary or eye infection.

10. A pharmaceutical composition comprising a therapeutically effective amount of a compound of claim 1, or a pharmaceutically acceptable salt, complex, or tautomer thereof, and a pharmaceutically acceptable carrier.
